(19) United States
(12) Patent Application Publication
(10) Pub. No.: US 2004/0038331 A1
(43) Pub. Date: Feb. 26, 2004

(54) Solid phase synthesis of biomolecule conjugates

(76) Inventors: M. Parameswara Reddy, Brea, CA (US); Firdous Farooqui, Brea, CA (US); Kurt L. Brillhart, Mission Viejo, CA (US)

Correspondence Address: PATENT LEGAL DEPARTMENT/A-42-C, BECKMAN COULTER, INC., 4300 N. HARBOR BOULEVARD, BOX 3100, FULLERTON, CA, 92834-3100, US

(21) Appl. No.: 10/227,107

(22) Filed: Aug. 23, 2002

(51) Int. Cl. C12P 21/06; C07K 5/00; C08B 37/00

(52) U.S. Cl. 435/68.1; 530/402; 536/55.3

(57) ABSTRACT

Processes for the solid state phase formation synthesis of biomolecule conjugates, particularly protein-oligonucleotide conjugates are shown. One of the protein or oligonucleotide is reversibly bound to a solid substrate phase. At least one portion of each of the protein and the oligonucleotide molecules is activated with complementary activation groups. The activated protein and the activated oligonucleotide are then reacted, in a buffered solution resulting in the formation of the desired conjugate which remains reversibly bound to the substrate. The nature of the buffered solution is then modified causing the conjugate to be released from the substrate solid phase.

This invention relates to the field of biochemistry. In particular, it sets forth a novel process for the solid phase synthesis of biomolecule conjugates.

### BACKGROUND

Protein-oligonucleotide conjugates have applications in the diagnosis of disease states, analysis of biological materials and as intermediates in the synthesis of biologically active compounds for therapeutic purposes. Specific examples include a) the generation of specific nucleic acids with specific proteins used for assay purposes, b) preparation of nucleic acid sequences which can be preferentially directed to specific protein recognition sites on specific cells as a result of a protein attached to the nucleic acid, and c) the ability to run multiplexed immunoassays in which an array of oligonucleotides hybridize specifically to oligonucleotide-antibody conjugates. Conjugates are typically produced by synthesizing the nucleic acid constituent and reacting it in solution with a protein, in combination with appropriate coupling chemistries. Extensive processing, typically involving chromatography, is then required to remove the unreacted starting materials and any undesirable end products

The art also includes numerous examples of the formation of peptide-oligonucleotide conjugates, polyamide-oligonucleotide conjugates, polyamide-protein conjugates and peptide-protein conjugates. However, the art does not show a simple and efficient process for producing protein-oligonucleotide conjugates, and the methods for forming other conjugates are not suitable for producing protein-oligonucleotide conjugates. In fact it was believed that the primary amino groups on synthetic oligonucleotides are protonated and unreactive under the low pH conditions necessary to activate protein carboxyl groups. As a result carbodiimide mediated conjugation of an amino derivatized oligonucleotide to a protein, while possible, proceeds only at a very low efficiency.

U.S. Pat. Nos. 5,525,465 and 5,677,440,to Haralambidis and Tregear describes the solid phase synthesis of short polypeptides, followed by the contiguous synthesis of oligonucleotide sequences beginning at the terminus of the peptide. The stated purpose of the peptide sequence is as a passive ‘tag’ for the oligonucleotide sequence, either through chemical modification of the amino acid side chains or by recognition with a peptide-specific antibody. This method is limited to synthetic peptides, the size of which are limited by the efficiency of each step of the synthesis. The literature suggests a maximum length of approximately 30 amino acids

U.S. Pat. No. 6,013,434 also to Tregear and Haralambidis, describes the synthesis of synthetic peptide-oligonucleotide conjugates utilizing a specific spacer between these two moieties. The spacer incorporates a modified ribose that permits the attachment of other molecules to the conjugate. This method is also limited to the use of small synthetic peptides, specifically through the carboxyl terminus. The particularly disclosed linkage between the peptide and the oligonucleotide permits the attachment of other potentially functional molecules.

U.S. Pat. No. 5,989,831 to Cros et al. describes the use of oligonucleotides as labeling groups, to be used as a ‘tag’ for small molecules in competitive immunoassays. It does not address the method by which oligonucleotides are conjugated to the molecules of interest. The scope is also limited to conjugation with small molecules, not large intact proteins.

U.S. Pat. No. 6,153,737 to Manoharan, Cook, and Bennett broadly covers the covalent linkage of an oligonucleotide to practically any compound with biological activity through a variety of sites and using a variety of linker molecules. The major reason for doing so is to increase the uptake of the oligonucleotide into the cell in order to regulate activity. The patent describes the nature of the covalent bond between the oligonucleotide and the other moiety; it does not address the mechanism by which that bond is formed. This patent is an example of a process whereby the protein and oligonucleotide are mixed together in solution, allowed to react for many hours, then separated by chromatographic techniques. In this instance the oligonucleotide must first be activated by incorporating a functional group at the 2′ position of the nucleotide.

U.S. Pat. No. 6,127,533 to Cook et al. describes the use of aminooxy nucleotides to form conjugates. The aminooxy moieties provide one or more conjugation sites useful for the conjugation of various ligands to the oligonucleotide. Subsequent to synthesis the amine groups can be used for the attachment of a large variety of molecules that enhance uptake of the oligonucleotide into cells, where it is intended to regulate activity.

U.S. Pat. No. 6,197,513 to Coull and Fitzpatrick, describes the use of synthetic PNA and DNA sequences that contain atypical, low pKa amines in standard coupling chemistries. The low pKa of these amines improves the efficiency of the coupling process by permitting it to occur at a pH where the other reactants are more stable. This patent requires the use of oligonucleotides containing specific nucleophilic groups.

Prior processes to conjugate biomolecules, in particular nucleic acids and/or proteins, with various different ligands have been dependent on solution-phase reactions followed by relatively inefficient and technically demanding separation steps to remove unreacted materials. The method described in this invention resolves many of these issues by reversibly binding one of the conjugate components to an insoluble phase, thereby greatly simplifying subsequent purification.

### BRIEF DESCRIPTION

Applicant has provided a novel method for synthesizing biomolecule conjugates, using reversible immobilization of one of the components and known reactions to activate selected sites on proteins and oligonucleotides and generate protein-oligonucleotide conjugates. The method described is not limited to the carboxyl terminus, as are some of the cited references, and uses different linker moieties from those described in the prior patents.

Described herein is a low cost, high efficiency, consistently repeatable process for reproducibly synthesizing biomolecule conjugates in high purity and high concentration. The process is simple, convenient, and requires little hands-on time. Skill in chromatography is not necessary. The process in a first embodiment includes, in part, reversibly binding a protein to a substrate, activating, in a controlled manner, one or more selected reaction sites on the protein, preparing an oligonucleotide having a single active site which will react with the activated site on the protein, the activated oligonucleotide being dissolved in a buffered salt solution, bringing the dissolved activated oligonucleotide into contact with the activated protein to form the desired conjugate, and releasing the conjugate so formed from the substrate. The buffered salt solution is selected so that the oligonucleotide remains in solution and does not disturb the binding of the protein to the substrate. As part of the process (to assure that only desired conjugate is produced) the substrate, bound protein, and formed conjugate are washed with the buffered salt solution after each step of the process to remove any unreacted material and leave only the intended product of each step of the process. The conjugate so formed is released from the substrate by washing with a solution chosen to weaken the interaction between the substrate and the conjugate without damaging the conjugate.

In a second embodiment, the oligonucleotide is first bound to or synthesized on a solid phase, hybridized with an activated complementary oligonucleotide, reacted with activated protein to form the conjugate and then the conjugate is release from the solid phase. The process includes: (1) washing the solid phase, bound oligonucleotide, and conjugate with a buffered carrier solution, (2) delivering the activated protein to the bound oligonucleotide, (3) releasing the formed conjugate using a solution of different concentration or pH which will weaken the interaction between the solid phase and the oligonucleotide-protein conjugate formed by the process.

### BRIEF DESCRIPTION OF THE DRAWINGS

FIG. 1 shows the iminothiolane reaction where P=protein

FIG. 2 shows the reaction of SMCC with amine containing molecule R (an oligonucleotide) and subsequent reaction with a thiol containing molecule R′ (a protein)

### DETAILED DESCRIPTION

Biomolecule conjugates are typically synthesized by separately derivatizing the components and mixing them together in a solution, usually with one component in large excess. This then must be followed by extensive processing, generally involving chromatography, to remove the unreacted materials and undesirable side products.

The process described herein is a simple process which comprises reversibly binding a first biomolecule reactant to a solid phase, the first reactant having a site thereon which is receptive to reaction, causing that site to react with a second biomolecule reactant in solution, the second reactant having a second reactive group thereon, thus forming the desired conjugate, and then washing the solid phase with bound conjugate (for example, a protein-oligonucleotide conjugate) with the solvent used to solubilize the second reactant. The desired biomolecule conjugate so generated can then be released from the solid phase for use in any manner intended. The result is a quantity of substantially pure biomolecule conjugate with substantially all of the conjugate molecules having the same intended chemical structure and biological activity. The first and second biomolecule can be selected from a broad selection of biomolecules which are reactive with each other or can be activate to so react. Examples of suitable biomolecules include, but are not limited to proteins, peptides, nucleic acids nucleotides, polynucleotides, oligonucleotides, carbohydrates and lipids. It is also contemplated that haptenic groups or labeling groups or other active groups can serve as the first or second biomolecule or can be added to the biomolecules. Examples of various haptenic groups include, but are not limited to drugs such as digoxin, Phenobarbital and theophylline. Examples of biomolecules to which these haptens can be added to provide an antibody response include bovine albumin and keyhole limpet hemocyanin. Examples of labeling groups which may be used or added include, but are not limited to fluorescent moieties, dyes, chemiluminescent or luminescent moieties and biotin or biotin analogs. Still further, other active groups, such as enzymes or chelating groups can be the first or second biomolecule and can function as labeling groups based on their measurable enzymatic or binding activity. Generally, labeling groups provide a predetermined and traceable functionality when part of a biomolecule conjugate. This activity can take a variety of forms. One implementation of this is to utilize a label that, via fluorescence, enzymatic production of a luminescent product, inherent radioactivity, etc. permits quantitation of the amount of biomolecule conjugate in a given area. A similar implementation is to utilize a label that, via specific intramolecular recognition such as a vidin-biotin, antibody-antigen, DNA duplex formation, etc. restricts the distribution of an attached biomolecule (and its attendant activity) to a specific area where such activity is desirable.

While the preferred process starts with a selected protein bound to an insoluble phase, followed by reaction with a desired oligonucleotide, the reverse, namely binding an oligonucleotide to the substrate followed by reaction with a protein is also within the scope of the patent. This is illustrated in formula 1; the process starting with an oligonucleotide bound to the substrate is shown in formula 2.

$$\begin{matrix}\begin{matrix}S : P \text{-} X + OLN \text{-} Y \to S : P \text{-} OLN \\ S : P \text{-} OLN \to S + P \text{-} OLN ( released )\end{matrix} \} & ( 1 ) \\ \begin{matrix}S \text{-} CS : OLN \text{-} Y + P \text{-} X \to S \text{-} CS : OLN \text{-} P \\ S \text{-} CS : OLN \text{-} P \to S \text{-} CS + OLN \text{-} P ( released )\end{matrix} \} & ( 2 )\end{matrix}$$

Where the

S=solid support

P=protein

CS=complementary sequence

OLN=oligonucleotide to be coupled

Y=a first reactive group

X=a second reactive group

and where the

S:P and CS:OLN bonds are readily reversible and

P—OLN and S—CS bonds are covalent

Formation of Conjugates Starting with a Bound Protein

Selection of substrate—The literature provides numerous examples of substrates to which proteins will readily bind. For example The Scientist, 16, (2): 40 (2002) and 37, Feb. 18, 2002 discusses techniques for purification of proteins. These techniques include, as a first step binding the protein to substrates, for example gels (in gel filtration procedures), ion exchange resins, hydroxyapatite and column materials used in hydrophobic interaction chromatography, high-performance liquid chromatography and affinity chromatograph, the list of such materials being incorporated herein by reference. The column materials used in hydrophobic interaction chromatography, high-performance liquid chromatography and affinity chromatograph are preferred. Suitable substrates are butyl Sepharose (cross-linked agarose beads preferably about 200μ in diameter), sulfopropyl sepharose, protein A sepharose and protein G sepharose and other materials typically used to pack affinity, liquid or gel chromatographic separation columns for separation of proteins. Numerous other matrices are usable including polymethylmethacrylate, crosslinked dextran, polystyrene, polyacrylamide, polymerized and/or crosslinked polyethylene glycol, controlled pore glass, and combinations thereof. However, these materials do not participate directly in the binding; they serve as a framework that binding groups are attached to. Suitable binding agents include: (1) almost any hydrophobic group (such as butyl, phenyl, octyl. hexyl, and propyl), (2) almost any charged group (such as sulfopropyl, diethylaminoethyl, carboxylate, and quaternary amines), (3) pseudo-specific groups such as aminophenylboronate, hydrophobic dyes used in dye affinity chromatography, enzyme cofactor and substrate analogs, certain sugars, and (4) specific proteins such as lectins, Fc receptors, antibodies to the protein of interest, etc. The substrate with binding agent is selected because of a specificity for binding to one biomolecule component of the desired conjugate and lack of attraction for other materials such as the second biomolecule component or activating reagents. When a protein dissolved in the appropriate buffered solvent is passed over or through an appropriate solid phase the protein becomes reversibly bound to it, based on an affinity of the protein for the solid phase and not necessarily due to covalent interaction.

Selection of Solvent and Protein—A buffered aqueous solution, which will not result in irreversible denaturation , such as phosphate buffer containing 1M Na₂SO₄ is used to dissolve the desired protein. A solvent having the same composition (referred to as the buffered solvent) is used in each of the steps detailed below. As long as the same composition of buffered solvent is used in each step, once the protein is attached to the substrate it will remain substantially attached until, as discussed below, the solvent composition is changed. Virtually any protein can be used. The process has particular utility for forming conjugates incorporating immunoglobulins, cyctochromes, receptors, enzymes, lectins, transport/storage proteins, recombinant proteins and virtually any macromolecule containing activatable chemical groups that has a reversible affinity for the solid phase.

Binding of Protein to Solid Phase—A substrate with adequate binding attraction for the selected protein is placed in a suitable retention vessel, such as a chromatography column, the protein is dissolved in the buffered solvent and the dissolved protein solution is passed over or through the solid phase. After liquid (solvent and unattached protein) is removed from the substrate, the solid phase is washed with quantities of fresh buffered solvent (protein free) to assure that all unbound protein has been removed.

Modification Of Bound Protein—Protein are comprised of amino acids linked into chains by amide bonds which result from the joining of the carboxyl group of one amino acid to the amino group of the next amino acid. Some proteins may also contain carbohydrates (glycoproteins) and lipids (lipoproteins). The numerous different amino acids or other constituents that can compose the protein include various different side chains. Amino acids found in proteins are listed in Table 1.

TABLE 1
AMINO ACIDS FOUND IN PROTEINS

Neutral Amino Acids - amino acids with unsubstituted side chains
Glycine
Alanine
Valine
Leucine
Isoleucine
Acidic Amino Acids
Aspartic Acid
Asparagine
Glutamic Acid
Glutamine
Basic Amino Acids
Arginine
Lysine
Hydroxylysine
Histidine
Hydroxyl Substituted Amino Acids
Serine
Threonine
Sulfur-containing Amino Acids
Cysteine
Methionine
Aromatic Amino Acids
Phenylalanine
Tyrosine
Tryptophan
Imino Acids
Proline
4-Hydroxyproline

It is imperative, in modifying the protein, that inherent protein functionality be preserved; this can be accomplished by modifying certain selected side chains on the substrate bound protein by adding a crosslinking agent. Exemplary sites which can be modified without changing protein functionality include, but are not limited to, the internally or terminal amine groups, such as in lysine or arginine; carboxyls, such as in glutamic or aspartic acid; thiols side chains in cysteine and reduced cystine; hydroxyls in serine, threonine, and carbohydrate side chains and aldehydes resulting from oxidation of naturally occurring carbohydrate side chains. Terminal or side chain nucleophilic sites on the protein may be activated by reacting with a bifunctional, homobifunctional or hetero-bifunctional crosslinking agent, reducing agent, or oxidizing agent.

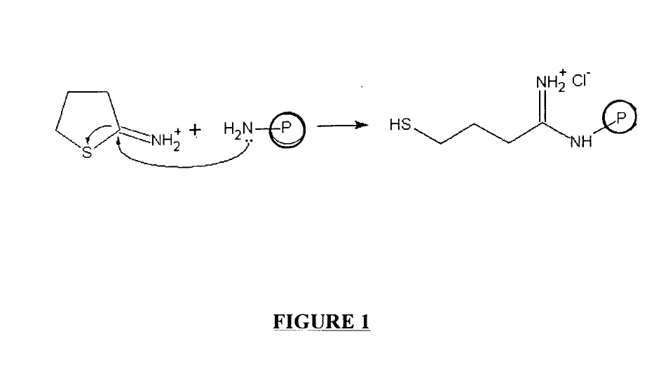

The agent selected to modify the protein is dissolved in the buffered solvent of the same composition and applied to the bound protein. After sufficient residence time the unreacted agent is washed from the substrate using clean buffered solvent until no more agent is removed. Alternatively, the protein may be modified prior to addition to the solid phase. In either case the activated protein remains bound to the substrate and excess reagents are removed by washing. FIG. 1 shows the reaction of iminothiolane with a protein to activate the protein.

Activation of Oligonucleotide—Nucleic acids, often referred to as polynucleotides or oligonucleotides, are composed of nucleotides that are chemically linked in specific linear sequences. The nucleotides are, in turn, composed of heterocyclic compounds (for example adenine (A), guanine (G), uracil (U), thymine (T) and cystosine(C)) bonded to phosphorylated forms of the sugars ribose and 2′-dioxyribose.

Oligonucleotides also have side and terminal groups or chains that can be activated with reagents which will in turn react with the activated sites on the protein. However, in doing so it is important that the base pair recognition is preserved. Sites on the oligonucleotide that can be modified, by addition of linking agents, with a reasonable expectation of preserving the base pair recognition for complementary strands include, but are not limited to a) amines or thiols added during nucleotide synthesis, b) naturally occurring terminal phosphate groups which can be reacted with carbodiimides and c) hydroxyls present on the deoxyribose or ribose groups, which can be activated using reagents such as cyanogen bromide, tresyl chloride, fluoromethylpyridine or triazine trichloride. The preferred site for activation is an amine; however thiol (—SH), —, —OH, and OPO₃⁻² may also be used. These groups are preferably terminal groups that, when reacted with the activated site on the protein leaving the oligonucleotide extending from the protein in contrast to side groups along the length of the oligonucleotide. It may also be necessary to protect other groups on the nucleotide so as to prevent unwanted reactions. Once the conjugate is formed the protecting groups can be removed.

The desired oligonucleotide can be synthesized using known techniques. In a preferred embodiment the substrate is polystyrene, Toyopearl™ (ethylene glycol/methacrylate copolymer) available from Tosoh Biosep, polymerized polyglycols, or controlled pore glass. Alternatively, the substrate, preferably Toyopearl, is obtained with a first nucleoside already attached via a protected amine linker on the 3′ hydroxyl. The 5′ hydroxyl is typically protected by addition of a dimethoxytrityl (DMT) group. The desired oligonucleotide is then synthesized on the substrate starting from the first attached nucleoside. To build the sequence the 5′ hydroxyl is deprotected with an acid and then reacted with an appropriate 3′ phosphoramidite nucleoside monomer, which also has a DMT protected 5′ hydroxyl. A capping step follows that acetylates any unreacted 5′hydroxyls. This assures that incorrect sequences will not be synthesized and facilitates removal of any “failure products”. The final step in the cycle is to convert the phosphite linkage into the more stable phosphotriester by oxidation with aqueous iodine. This procedure is then repeated to gradually build the desired sequence. The completed sequence can then be released by exposing the support to ammonium hydroxide, which also removes any protecting groups from the bases. The released amino-oligonucleotide is then purified by HPLC. This oligonucleotide can then be activated and conjugated to a protein bound to a surface as described herein. Prefered oligonucleotides typical contain 5 to 60 nucleotide units, referred to as a 5 mer to 60 mer oligonucleotides.

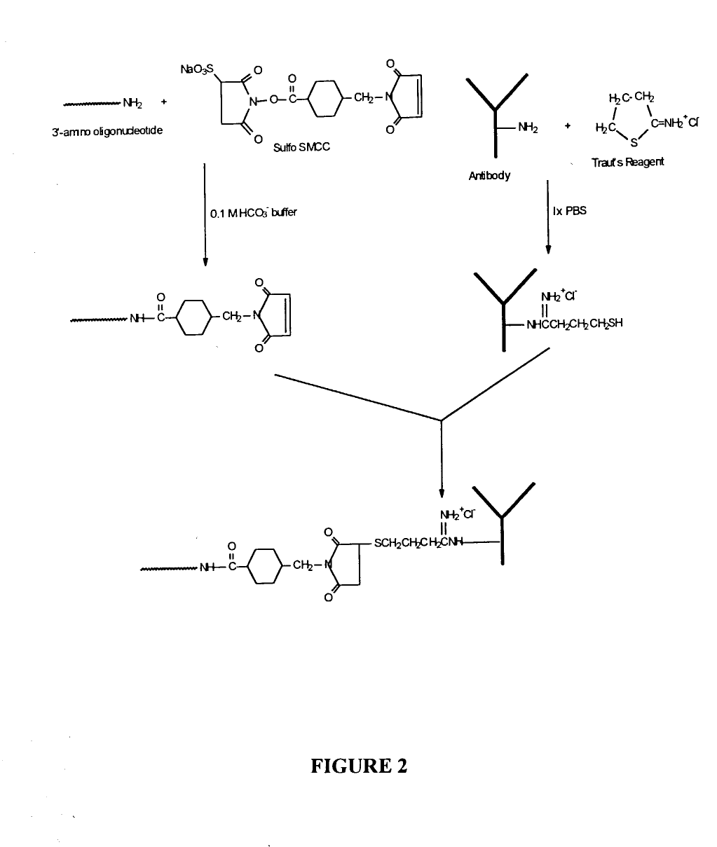

To prepare the oligonucleotide for conjugation with the protein the oligonucleotide is activated by reacting with a bifunctional, homobifunctional or hetero-bifunctional crosslinking, reducing agent or oxidizing agent. FIG. 2 shows the SMCC activation of an amine containing oligonucleotide and the subsequent reaction with a thiol containing iminothiolane modified protein. Other crosslinking agents, such as SIA and DSS can also be used. A wide variety of crosslinking agents that could be adapted to the process are available through commercial sources.

Forming The Conjugate—Once the activated oligonucleotide is formed and washed to remove all unreacted and undesired side product, it is placed in the buffered solvent and brought into contact with the bound, activated protein to form the desired conjugate, which remains bound to the substrate. It has been found that 1 to 4 (or more) of the same oligonucleotides can be attached to each bound protein. The bound conjugate is rinsed thoroughly with fresh buffered solvent. When the wash solution no longer contains any dissolved material the conjugate can be released from the solid phase. This is accomplished by washing with an aqueous solvent having a different buffering agent, a higher or lower salt concentration or a different pH. For example, the pH can be reduced to 3.5 or increased to 9 in contrast to a neutral pH in the buffered solvent. Alternatively, the salt concentration (1M Na₂SO₄ in the buffered solvent) can be significantly reduced or a salt free solution can be used. A still further method is the addition of a large excess of a free binding group, for example, adding an excess of the correct sugar required to release a bound lectin-oligo conjugate. As shown in FIG. 2, the end result is a relatively pure conjugate.

Formation of Conjugates Starting with a Bound Oligonucleotide

In a manner similar to that described above a conjugate can be formed by first binding an oligonucleotide to a substrate, reacting an activated protein with certain activated sites on the oligonucleotide to form a bound conjugate and then releasing the conjugate from the substrate. It is preferred to use sequence-specific hybridization to conjugate proteins to bound oligonucleotides. However, anion exchange or other functionalities may be used.

In one embodiment, a primary oligonucleotide covalently attached to a substrate, or formed in situ as described above attached to a substrate as desired, is provided. A secondary oligonucleotide is bound by allowing it to hybridize to a complementary sequence synthesized as described above. The primary oligonucleotide is not released from the solid phase. The secondary oligonucleotide typically has a terminal amine group, which is activated while it is hybridized to the primary oligonucleotide on the solid phase. A neutral or slightly alkaline solution of NaCl≦1M is used to rinse the bound oligonucleotide as it will preserve the DNA duplex. A desired protein, typically a natural protein, is activated in a manner such as described above or by other known techniques so that it will react with the modified terminal amine group on the oligo-nucleotide. The activated protein is then washed with the same composition of salt solution to remove any unreacted material and then placed in contact with bound oligonucleotide for about 4-12 hours. This results in an oligonucleotide-protein conjugate bound to the substrate. After washing the bound conjugate with more of the same salt solution (until no more material is removed) the bound conjugate is released from the substrate by washing with a different concentration solution, or a different pH solution, or a differently buffered solution, or pure water. The end result is a conjugate of an oligonucleotide with a protein. Alternatively, the amino-oligonucleotide may be bound to an anion exchange matrix and a similar series of reactions carried out at low ionic strength, the final conjugate being released by increasing the buffer's ionic strength.

The conjugate produced may be the same irrespective of whether the starting material is a bound protein or a bound oligonucleotide. However, it is possible within the scope of the procedure described herein to attach more than one oligonucleotide, and they may be the same or different nucleotides, to a single protein. Alternatively, the process described herein may be used to attach more than one of the same protein. Also, multiple different proteins can be bound to an oligonucleotide if the oligonucleotide contains a number of activated groups. Typically, the process uses an oligonucleotide with a single amine group. However, within the scope of the process starting with a bound protein, more then one site may be activated on the protein, which will allow a nucleotide to be attached to each of the activated sites on the protein. In the same manner, starting with an oligonucleotide bound to a substrate, more then one site may be activated allowing more than one protein to be attached thereto. Also, where more than one site on the protein or oligonucleotide is activated, the various activated sites may be activated in the same or a different manner so that each particular active site will react with a particular selected protein or oligonucleotide, as the case may be.

As an example, the protein can be modified to have up to ten thiol groups which then enhances the rate and efficiency of the reaction. Each of these thiol groups can bind a single activated oligonucleotide. The number of oligonucleotides per protein in the final product, determined by UV absorbance, usually results in 2 or more oligonucleotides per IgG. It is desirable to react remaining thiol groups on the conjugate with reagents such as N-ethylmaleimide or iodoacetamide following conjugation to prevent crosslinking via disulfide formation.

Using the same techniques described herein oligonucleotide-peptide, oligonucleotide-enzyme and oligonucleotide-receptor conjugates can be prepared.

### EXAMPLE 1

### Procedure for Solid-Phase Synthesis of Immunoglobulin-Oligonucleotide Conjugate Using Immunoglobulin Bound to Butyl HIC Media

Materials:

1 mL Butyl Sepharose 4 Fast Flow HIC media (Pharmacia-Amersham)

Poly-Prep column (BioRad, P/N 773-1550)

NAP-25 desalting column (Pharmacia-Amersham) IgG

Iminothiolane

Oligonucleotide (30 mer) (AAGGCCACGTATTTTGCAAGCTATTTAACT such as shown in U.S. Pat. No. 5,648,213).

Sulfo-SMCC (Sulfosuccinimidyl-4-(N-maleimidomethyl) cyclohexane-1-carboxylate)

20 mM phosphate+1M Na₂SO₄, pH 7.5 (binding buffer)

20 mM phosphate, pH 7.5 (elution buffer)

0.1M NaHCO₃, pH 8.5 (bicarbonate buffer)

Procedure:

1. 2 mg of sulfo-SMCC are added to 32 A_{260 nm} of the amino-oligonucleotide in 1 mL of 0.1M bicarbonate buffer and the mixture is incubated for 1 hour at room temperature. In this context 1 A_{260 nm} is the amount of oligonucleotide required to give an absorbance of 1 AU at 260 nm when dissolved in 1 mL of water and measured with a 1 cm path length.

2. Unreacted sulfo-SMCC is removed from the reaction mixture using a NAP25 desalting column equilibrated with binding buffer as follows:

(a) the column is equilibrated with 25 mL of binding buffer

(b) the reaction mixture (1 mL) from step 1 is added and allowed to completely enter the column

(c) 1.5 mL of binding buffer is added and allowed to flow through the column

(d) 2 mL binding buffer is then added to the column and the eluted material is collected.

3. 1 mL of butyl HIC media is added to a PolyPrep column and rinsed with several mLs of water to remove the ethanol storage solution. It is then equilibrate with 10 mL of binding buffer.

4. 2 mg of IgG in approximately 1 mL of binding buffer is added to the butyl column and is allowed to flow through, followed by 4 mL of binding buffer. The liquid flowing through is retained and the absorbance at 280 nm is measured to verify binding of the IgG to the column. This step may be repeated if binding is not complete. A tip closure is then applied to the column.

5. A 2 mg/mL stock solution of iminothiolane is prepared in the binding buffer. 80 μL is added to 2 mL of binding buffer and it is poured into the column. The column is then capped and tumbled for 1 hour at room temperature.

6. Any unreacted iminothiolane is removed and the column is washed with 20 mL of binding buffer. The tip closure is rinsed thoroughly and reapplied to the column.

7. The oligo-SMCC eluted in step 2d above is added to the column, the column is recapped (the cap is rinsed thoroughly), and tumbled overnight at room temperature.

8. 2.5 mg of dry N-ethylmaleimide is added to the column and tumbled for 1 hour at room temperature to block residual thiol groups on the antibody.

9. Any unreacted oligonucleotide is removed by washing the column with binding buffer until the absorbance at 260 nm drops below 0.01 AU.

10. The IgG-oligonucleotide conjugate which is now bound to the insoluble phase is released by passing 4 mL of elution buffer through the butyl sepharose column.

The formation of the conjugate was confirmed by CE or PAGE analysis.

### EXAMPLE 2

### Solid-Phase Synthesis of Oligonucleotide-Antibody Conjugates Using Protein A-Sepharose

A 1 mL column of protein A-sepharose (Amersham Biotech) is prepared and equilibrated with 20 mM phosphate+3M NaCl, pH7.5. Two mg of mouse IgG is recirculated through the column at 1 mL/min until all of the protein is bound. A solution of 20 mM phosphate+3M NaCl+1 mM dithiothreitol (DTT) is then recirculated through the column for 1 hour at room temperature in order to convert some of the IgG disulfide bonds to thiol groups. Protein A does not contain disulfide bonds and is therefore not affected by this procedure. Subsequent to DTT treatment the column is washed extensively with 20 mM phosphate+3M NaCl. 32 A_{260 nm} of Sulfo-SMCC treated oligonucleotide in 1 mL of 20 mM phosphate+3M NaCl is recirculated through the column overnight at room temperature, after which unreacted oligonucleotide is removed by extensive washing in this buffer. Conjugate is released by passing several mL of 20 mM glycine, pH 3.0, through the column. The formation of the conjugate was confirmed by CE or PAGE analysis.

### EXAMPLE 3

### Solid Phase Synthesis of Oligonucleotide-Antibody Conjugates Using Sulfopropyl Fast Flow Ion Exchange Media

One mL of Sulfopropyl Fast Flow ion exchange media (Amersham Biotech) is placed in a small disposable column and equilibrated with 20 mM Na Acetate, pH 6.0 Excess buffer is removed and 2 mg of mouse IgG in the same buffer is passed repeatedly over the column until all protein is bound. Excess buffer is removed and 32 A_{260 nm} of sulfo-SMCC activated oligonucleotide in 1 mL of acetate buffer is added to the column. Both ends are capped and the column tumbled for 48 hours at room temperature. After extensive washing of the solid phase with acetate buffer to remove unreacted oligonucleotide the conjugate is released by flowing several mLs of 100 mM Tris+1 M NaCl, pH8, through the column. The formation of the conjugate was confirmed by CE or PAGE analysis.

### EXAMPLE 4

### Solid-Phase Synthesis of Oligonucleotide-Enzyme Conjugates Using Butyl Sepharose

A 1 mL column of butyl-sepharose (Amersham Biotech) is prepared and equilibrated with 20 mM phosphate+1M Na₂SO₄, pH7.5. One mg of horseradish peroxidase (Type V, Sigma Chemical Company) in 1 mL of the equilibrating buffer is added to the column and allowed to flow through. Binding of the enzyme is verified by monitoring the UV absorbance of the flowthrough. One mL of 10 mM iminothiolane in 20 mM phosphate+1M Na₂SO₄, pH7.5 is added to the column, which is then capped and tumbled for 1 hour at room temperature. The support is then thoroughly washed by uncapping the column and passing 20 mL of 20 mM phosphate+1M Na₂SO₄, pH7.5 through the gel bed. 16 A_{260 nm} of Sulfo-SMCC treated oligonucleotide in 1 mL 20 mM phosphate+1M Na₂SO₄, pH7.5 is added to the column, which is then capped and tumbled overnight at room temperature. Uncoupled oligonucleotide is then removed by extensive washing in this buffer. Conjugate is released by passing several mL of 20 mM phosphate, pH 7.5, through the column. The formation of the conjugate was confirmed by CE or PAGE.

### EXAMPLE 5

### Solid Phase Synthesis of Oligonucleotide-Antibody Conjugates Using Solid Phase Oligonucleotide Hybridization

A first oligonucleotide (Oligo 1) is synthesized attached to an Oligo Affinity support (5′ Dimethoxytrityl-Adenosine-2′,3′ diacetate-N-Linked-CPG, Glen Research part # 20-4001-01) on 1 μmole scale on ABI 394. The attached oligo is deprotected using concentrated ammonia at 55° C. for 5 hours. The support with attached oligo is then washed with water (3×2 ml).

10 A_{260 nm} of an amino oligo complementary to the first oligo (e.g. Oligo 1′) is reacted with 1.2 mg of sulfo SMCC in 1001 μl of 0.1 M NaHCO₃ buffer (pH 8.2) and shaken at room temperature for 1 hour to form on SMCC oligo.

The SMCC oligo is added to the first oligo attached in trishydroxyethylamine (Tris) buffer with the final concentration of the mixture being 0.1 M Tris, pH 7.5, +3M NaCl and shaken at 37° C., overnight.

The support is brought to room temperature for about 1 hour; the support is then washed with 1M NaCl and the supernatant is viewed at 260 nm until the washes have 260 nm absorbance of 0.001 (approximately 5×1 ml)

3 mg of an antibody (mouse IgG) is activated with 75 μL of iminothiolane (2 mg/ml solution) in 300 μL of PBS and shaken at room temperature for 1 hour to form an activated antibody solution. Excess iminothiolane is then removed by chromatography on Sephadex G25.

The activated antibody solution is added to the oligo attached to the solid support with final concentration of the solution being 2 M NaCl+2 mM EDTA in PBS (Phosphate Buffered Saline—20 mM phosphate+150 mM NaCl, pH 7.4).

The solid support is shaken for 24-48 hours with activated antibody solution at room temperature. The support is washed with 1M NaCl to remove the unreacted antibody until the absorption at 280 nm is 0.02. (˜4×1 mL).

The oligo-antibody conjugate is released from the substrate with 10% by volume ethanol/water by heating the solution at 37° C. for 1 hour then rapidly cooling it. This process is repeated with 2×1 ml of 10% ethanol/water until the absorption at 260 nm-280 nm ratio is 1-1.5.

The formation of the conjugate was confirmed by CE or PAGE analysis.

It is evident from the foregoing that there are many additional embodiments of the present invention, which, while not expressly described herein, are within the scope of this invention and may suggest themselves to one of ordinary skill in the art. It is therefore intended that the invention be limited solely by the appended claims.

We claim:

1. A method of forming biomolecule conjugates comprising:
a. reversibly binding a first component to an insoluble phase, the first component comprising a biomolecule having one or more reactive chemical groups,
b. providing a second component in a liquid carrier, the second component being a biomolecule with one or more reactive chemical groups, the second component not binding to the insoluble phase,
c. applying the second component to the first component bound to the insoluble phase, said first and second components being maintained in contact with each other for a sufficient time to allow the reactive chemical groups on the first and second components to react to form a conjugate, the conjugate remaining bound to the insoluble phase,
d. removing unreacted materials by washing the insoluble phase with bound conjugate using a solvent that does not disrupt conjugate binding to the insoluble phase, and
e. releasing and collecting the biomolecule conjugate so formed from the insoluble phase by eluting with a solvent that reverses the binding of the first component established in step (a) above,

2. A method of forming biomolecule conjugates comprising:
a. reversibly binding a first component to an insoluble phase, the first component comprising of a biomolecule with one or more reactive chemical groups,
b. providing a second component in a liquid carrier, the second component being a biomolecule having one or more reactive chemical groups, the second component not binding to the insoluble phase,
c. activating a portion of the first component and/or a portion of the second component such that the first component and the second component will react by way of the activated portion or portions when brought into contact to form a conjugate,
d. bringing the second component into contact with the bound first component, said first and second components being maintained in contact with each other for a sufficient time to allow the activated portions on the first and second components to react to form a conjugate, the conjugate remaining bound to the insoluble phase,
e. removing unreacted materials by washing the insoluble phase with bound conjugate using a solvent that maintains conjugate binding to the insoluble phase, and
f. releasing and collecting the conjugate so formed from the insoluble phase by eluting with a solvent that reverses the binding established in step (a).

3. The method as described in claim 1, wherein the first component and the second component are selected from the group consisting of a protein, peptide, nucleic acid, polynucleotide, oligonucleotide, nucleotide, carbohydrate, lipid, haptenic group, or labeling group.

4. The method as described in claim 3, wherein the labeling group consists of a fluorescent moiety, dye, chemiluminescent moiety, luminescent moiety, or biotin or biotin analogs.

5. The method of claim 3 wherein the first or second component is an enzyme or a chelating agent.

6. The method as described in claim 3, where the first component is an immunoglobulin.

7. The method as described in claim 3 where the first component is an enzyme

8. The method as described in claim 3 where the second component is an oligonucleotide

9. The method as described in claim 7 where the final product is an antibody-oligonucleotide conjugate.

10. The method as described in claim 7 where the final product is an enzyme-oligonucleotide conjugate.

11. The method of claim 1 wherein the insoluble phase is polymethacrylate, sepharose compounds, cross-linked agarose, cross-linked dextran, polyacrylamide, cross-linked polyethylene glycol, polystyrene, controlled pore glass, or a combination thereof suitable to provide reversible binding of the first component and the conjugate so formed.

12. The method of claim 3 where the first component is a protein and terminal or side chain nucleophilic sites on the protein are activated by reacting with a bifunctional, homobifunctional or heterobifunctional crosslinking agent, reducing reagent, or oxidizing reagent.

13. The method of claim 3 wherein the second component is an oligonucleotide and terminal or base nucleophilic or electrophilic sites on the oligonucleotide are activated by a bifunctional, homo bifunctional or hetero bifunctional crosslinking agent, reducing reagent, or oxidizing reagent.

14. The method of claim 1 wherein the reversibly bound first component and insoluble phase are washed with a buffer solution, the second component in the buffered solution is brought into contact with the reversibly bound first component, and after each step of the process the product generated by each step of the process is thoroughly washed with a clean aliquot of the buffered solution.

15. The method of claim 14 wherein the buffered solution is selected so as not to disturb the binding between the first component and the insoluble phase.

16. The method of claim 15 wherein the buffered solution is selected from the group consisting of a Na₂SO₄ solution, a NaCl solution with a phosphate buffer, a bicarbonate solution, a sodium acetate solution and a tris solution.

17. The method of claim 12 wherein the bound conjugate is released from the insoluble phase by washing with a solution of a different ionic strength, pH, dielectric poin or competing ligand.

18. The method of claim 1 wherein the first component is a protein and the second component is an oligonucleotide, the first component and second component forming a substrate-bound protein-oligonucleotide conjugate.

19. The method of claim 18 wherein the substrate-bound protein-oligonucleotide conjugate is released from the substrate solution by washing the bound conjugate with a solution of the same buffered pH but having a different salt concentration.

20. The method of claim 3 wherein the oligonucleotide is a 5 mer to a 60 mer.

21. The method as described in claim 2, wherein the first component and the secondcomponent are selected from the group consisting of a protein, peptide, nucleic acid, polynucleotide, oligonucleotide, nucleotide, carbohydrate, lipid, haptenic group, or labeling group.

22. The method as described in claim 2, wherein the labeling group consists of a fluorescent moiety, dye, chemiluminescent moiety, luminescent moiety, or biotin or biotin analogs.

23. The method of claim 2 wherein the first or second component is an enzyme or a chelating agent.

24. The method of claim 2, where the first component is an immunoglobulin.

25. A method of forming a protein-oligonucleotide conjugate comprising:
a) reacting an excess of sulfoSMCC with an oligonucleotide in a buffered solution to form a mixture containing activated oligonucleotide,
b) removing any unreacted sulfoSMCC by passing the mixture through a desalting column to produce a clean activated oligonucleotide,
c) adding a protein in a binding buffer solution to a support media contained in a column to produce a substrate with bound protein,
d) reacting an activation compound with the bound protein to produce an activated, bound protein,
e) adding activated oligonucleotide to the column containing the activated, bound protein, while maintaining in contact with the protein for a period of time sufficient to form a bound conjugate, and
f) removing the bound conjugate from the column by addition of an elution buffer.

26. The method of claim 25 wherein the buffered solution is selected from the group consisting of 20 mM phosphate in 3M NaCl, 1M Na₂SO₄ at a pH of about 7.5, or a 20 mM Na Acetate solution at a pH of about 6.0

27. The method of claim 25 wherein the oligonucleotide is prepared in a 0.1M bicarbonate buffer solution.

28. The method of claim 25 wherein after each step of the method the prior prepared material is washed with a fresh aliquot of the same buffered solution to remove any unreacted material.

29. The method of claim 25 wherein the protein is IgG.

30. The method of claim 25 wherein the elution buffer is selected from a 20 mM phosphate solution, without any NaCl or Na₂SO₄, at a pH of about 7.5, 20 mM glycine at a pH of 3.0, or 100 mM Tris with 1M NaCl at a pH of about 8.

31. The method of claim 25 wherein the support media is selected from butyl HIC, protein A-sepharose, or sulfopropyl ion exchange media.

32. The method of claim 25 wherein the activation compound is iminothiolane or dithiothreitol.

33. A method of forming a oligonucleotide-protein conjugate comprising:
a) forming an activated oligonucleotide reversibly bound to an insoluble phase by, alternatively, hybridization to a complementary oligonucleotide covalently attached to the insoluble phase or by interaction directly the with the insoluble phase,
b) activating a protein and reacting the activated protein with the activated oligonucleotide reversibly bound to the insoluble phase to form a reversibly bound oligonucleotide-protein conjugate,
c) releasing the oligonucleotide-protein conjugate from the insoluble phase

34. The method of claim 33 wherein the protein is an antibody and/or immunoglobulin.

35. The method of claim 33 wherein the protein is activated with iminothiolane.

36. The method of claim 33 wherein the protein is in a buffered solution comprising 2M NaCl, 2 mM EDTA and PBS.

37. The method of claim 33 wherein after each step of the method the prior prepared material is washed with a fresh aliquot of the same buffered solution to remove any unreacted material.

38. The method of claim 33 wherein the oligonucleotide-protein conjugate is released from the substrate using 10% by volume ethanol/water.

39. The method of claim 33 where in the support media is polymethacrylate, sepharose, cross-linked agarose, cross-linked dextran, polyacrylamide, cross-linked polyethylene glycol, polystyrene, controlled pore glass, or combinations thereof.

40. The method of claim 25 where in the support media is polymethacrylate, sepharose, cross-linked agarose, cross-linked dextran, polyacrylamide, cross-linked polyethylene glycol, polystyrene, controlled pore glass, or combinations thereof.
